(19) United States
(12) Patent Application Publication
(10) Pub. No.: US 2010/0221818 A1
(43) Pub. Date: Sep. 2, 2010

(54) Device for the extemporaneous preparation of active preparations

(75) Inventors: Philippe BORDEAU, Meyzieu (FR); Martin GUILLAUME, Nantua (FR); Marc COLLADO, Lyon (FR); Barbara LENOIR, Saint Quentin Fallavier (FR)

Correspondence Address: OLIFF & BERRIDGE, PLC, P.O. BOX 320850, ALEXANDRIA, VA, 22320-4850, US

(73) Assignee: ALAXIA, VAULX EN VELIN (FR)

(21) Appl. No.: 12/695,752

(22) Filed: Jan. 28, 2010

(30) Foreign Application Priority Data
Jan. 28, 2009 (FR) 0950539

(51) Int. Cl. C12M 1/00; B01J 19/00

(52) U.S. Cl. 435/289.1; 422/224; 422/187

(57) ABSTRACT

The invention relates to an autonomous portable device for the storage of at least two formulations and for the automatic extemporaneous preparation of an active solution from the two formulations, said device comprising: a first compartment for containment of a first formulation, a second compartment for containment of a second formulation, a retaining chamber which communicates at least with the first compartment by means of at least one transfer channel and which comprises at least one outlet channel for the active solution, manually triggered mixing and expelling means suitable for, after triggering, automatically: bringing the two formulations into contact, and expelling, via the outlet channel, at least a part of the active solution at the end of a minimum reaction time.

The present invention relates to a device for extemporaneous preparation of solutions containing active agents, that can be used in the human or veterinary pharmaceutical field, in the diagnostics or analysis field and, more generally, for the extemporaneous preparation of a formulation that requires two formulations to be brought into contact with one another.

It relates more particularly to a device for the preparation of therapeutic doses of a hypothiocyanite and/or hypohalite ion solution.

The hypothiocyanite and/or hypohalite ion is generated in particular in vivo by the lactoperoxidase system according to the equation below:

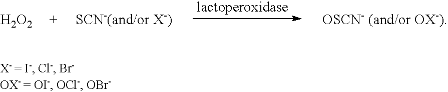

The pharmacological properties of the hypothiocyanite ion and of hypohalite ions are known, in particular their biocidal and/or bacteriostatic and/or virucidal properties. However, owing to the instability of this chemical species, the half-life of which, in the absence of peroxidase and/or of hydrogen peroxide, is approximately 24 hours for the hypothiocyanite, the methods of administration envisioned thus far were methods of administration enabling them to be generated in vivo and therefore containing the enzymatic generator(s), namely lactoperoxidase and/or glucose oxidase, or even the administration of precursor species, namely thiocyanate ions or halides.

WO2007134180, for example, discloses a therapeutic composition which acts through the action of the hypothiocyanite ion, comprising an enzyme system, an oxidoreductase which, by reduction of a specific substrate, for example glucose, produces hydrogen peroxide, the specific substrate, namely the thiocyanate ion (SCN⁻) and lactoperoxidase. The difficulty in formulating such therapeutic compositions and the side effects that may be generated, for example, by the in vivo production, in this case in the respiratory tracts, of hydrogen peroxide, can be understood.

In US 2002/172645 (E. G. Conner), the thiocyanate ion is administered alone in order to feed the endogenous lactoperoxidase system and to form hypothiocyanite ions in vivo, or can be administered in combination with the lactoperoxidase system.

None of the solutions developed thus far has enabled the administration of this very active chemical species under satisfactory conditions.

In addition, WO 02097076 discloses a system for industrial production of the hypothiocyanite ion, for example for the production of solutions intended for the treatment of food products. The solutions obtained cannot, however, be used for the formulation of pharmaceutical compositions owing to the half-life of the active ionic species.

The hypothiocyanite and/or hypohalite ions are obtained through the action of an enzymatic catalysis agent, for example a peroxidase-type enzyme, for example lactoperoxidase, on an aqueous-phase oxidizable substrate, halogen or pseudohalogen, capable of giving the hypothiocyanite and/or hypohalite ion, in the presence of an oxygen donor, for example hydrogen peroxide.

The oxygen donor may also be in the form of a supplementary enzyme system which produces oxygen peroxide and which comprises an oxidizable substrate and an enzyme, for example of the oxidoreductase type, specific for this substrate, for example glucose in the presence of glucose oxidase.

The present invention has the objective of solving the abovementioned problems and enables a user, without particular skill, to autonomously produce an extemporaneous preparation of a sufficient amount of a daily dose.

Thus, the invention relates to an autonomous portable device for the storage of at least two formulations and for the automatic extemporaneous preparation of an active and/or purified solution by reaction of the two formulations.

According to the invention, the autonomous portable device comprises:
a first compartment for containment of a first formulation,
a second compartment for containment of a second formulation,
a retaining chamber which communicates at least with the first compartment by means of at least one transfer channel and which comprises at least one outlet channel for the medicinal solution,
manually triggered mixing and expelling means suitable for, after triggering, automatically:
bringing the two formulations into contact,
and expelling, via the outlet channel, at least a part of the medicinal solution obtained at the end of a minimum reaction time.

In one embodiment, at least one of the formulations is in solid form. In this embodiment, the formulation may, for example, be in the form of powder or of freeze-dried product; it may contain one or more active agents that are stable in said form.

In one embodiment, at least one of the formulations is in liquid form. In this embodiment, the formulation may, for example, be a solution of one or more active agents, stable in this form, but also a solvent, physiological saline, an isotonic solution, water or a buffer.

The use of separate containment compartments makes it possible to effectively isolate the first and the second formulation, preventing interaction thereof during the device storage period and therefore reaction thereof. Thus, the first and the second formulation can be stored in the device according to the invention in stable forms suitable for conservation and for storage for several weeks, or even several months, without any risk of their active properties being affected by this storage.

Furthermore, the use of the manually triggered mixing and expelling means which automatically bring the two formulations into contact and then provide expulsion at the end of a minimum reaction time enables the preparation of an active solution under good conditions while in particular adhering completely to the reaction kinetics so as to guarantee that the solution expelled has a sufficient amount of active agent to provide the desired effect.

In one embodiment, the first formulation in solution comprises at least one aqueous-phase oxidizable substrate capable of giving the hypothiocyanite and/or hypohalite ion.

In another embodiment, the first formulation is a liquid solution which will make it possible to solubilize the second formulation in the form of powder, for example a freeze-dried product, in a chosen amount of time.

When the oxygen-donor system is in the form of a supplementary enzyme system, the first formulation in solution further comprises at least one reducible substrate.

The solid or liquid second formulation comprises at least one enzymatic catalysis agent and, when the oxygen donor system is in the form of a supplementary enzymatic system, at least said supplementary enzymatic system.

In one particular embodiment, the oxidizable substrate is constituted of at least thiocyanate or halide ions and the second formulation comprises at least one lactoperoxidase.

In one embodiment, the reducible substrate is glucose.

In one embodiment, the supplementary enzyme system is a glucose oxidase.

According to the invention, the autonomous portable device can be produced in various ways. Thus, it may be envisioned that the two compartments intended for storage, respectively, of the first and of the second formulation are arranged side by side and run in parallel to one another into the retaining chamber, each by means of a corresponding transfer channel. The formulations are then each expelled from their respective compartment by a piston propelled, for example, by a mechanical or electromechanical system powered by one or more small storage battery power sources.

According to one embodiment, the autonomous portable device in accordance with the invention comprises:
an elongated tubular body which comprises, at a distal end, a head in which the transfer channel is fitted and, on the opposite side from the head, at a proximal end, a filling aperture which partly delimits at least the first compartment,
a hole-punched spacer which is placed inside the tubular body, remote from the distal end, and which delimits the second compartment closed off, at opposite faces of the spacer, by two cleavable protective seals,
   and as mixing and expelling means:
a hollow body which delimits the retaining chamber, which is connected to the transfer head and comprises, on the opposite side from the transfer head, an outlet head in which the outlet channel is fitted,
and as a mixing and expelling means:
a piston which can move translationally in the tubular body between a resting position and a working position and which bears, on the one hand, on a face directed toward the spacer and the distal end of the body, a perforating finger intended to perforate the protective seals of the second compartment, and, on the other hand, on a face directed toward the proximal end, a locking rod,
a spring which is interposed between the piston and a maintaining cap rigidly connected to the body in such a way as to push the piston toward its working position,
a locking means.

In one embodiment, said locking means is a removable locking tongue which is interposed between the locking rod and the cap in such a way as to maintain the piston in the resting position.

In one embodiment, said locking means is a removable cover which masks the locking rod and the cap in such a way as to maintain the piston in the resting piston.

The use, as expelling and mixing means, of a piston actuated by a spring makes it possible to obtain the automatic operation and the autonomy of the extemporaneous preparation device in a particularly simple and inexpensive manner. In order to guarantee that the reaction kinetics are respected, the compression of the spring in the resting position of the piston, the stiffness of the spring, the stroke of the piston, the passage section of the transfer channel and the volume of the retaining chamber are adapted such that the expulsion of the active solution occurs after a period of time necessary, after unlocking, for the reaction between the two formulations to be complete.

In one embodiment, this period of time is between 1 and 10 minutes, and for example 5 minutes after unlocking.

According to one characteristic of the invention, the device may comprise a filter placed in the retaining chamber, being interposed between the transfer and outlet channels such that the filter provides filtration that can be described as frontal. The use of such a filter is particularly advantageous when one and/or the other of the two formulations comprises solid particles that may be an obstacle to optimal use of the active solution obtained. According to one characteristic of the invention, the filter may be suitable for providing an ultrafiltration below 500 kD, or even below 200 kD, or even below 70 kD. The filter may thus be suitable for providing an ultrafiltration at 50 kD, or even at 30 kD or at 10 kD.

According to one characteristic of the invention aimed at providing an optimal filtration, the spring may be suitable for exerting a pressure, measured at the internal face of the filter, of between 1 and 7 bar, and in one embodiment, greater than 5 bar.

Of course, the various characteristics and embodiments of the autonomous portable device according to the invention are capable of being combined with one another according to various combinations, insofar as they are not incompatible or exclusive with respect to one another.

Moreover, various other characteristics and advantages of the autonomous portable device according to the invention will emerge from the description below, given with reference to the drawings which illustrate a nonlimiting embodiment of an autonomous portable device according to the invention.

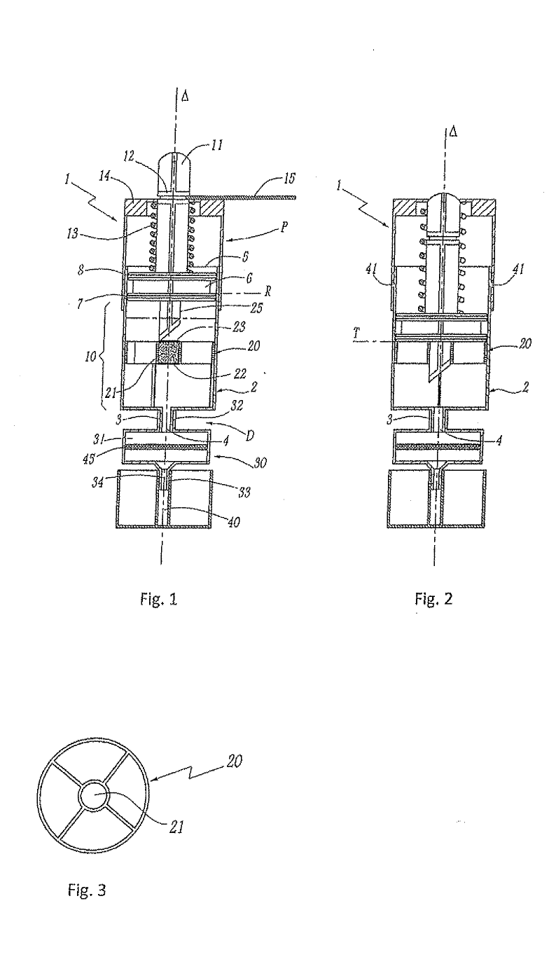

FIG. 1 is a diagrammatic axial section of an autonomous portable device for the automatic extemporaneous preparation of an active solution from two formulations, this device being in accordance with the invention and illustrated in a resting or storage position.

FIG. 2 is a section substantially equivalent to FIG. 1, showing the autonomous portable device according to the invention in the working position at the end of the sequence for preparing the active solution.

FIG. 3 is an elevation, viewed from above, of a spacer which is a constituent of the autonomous portable device as illustrated in FIGS. 1 and 2.

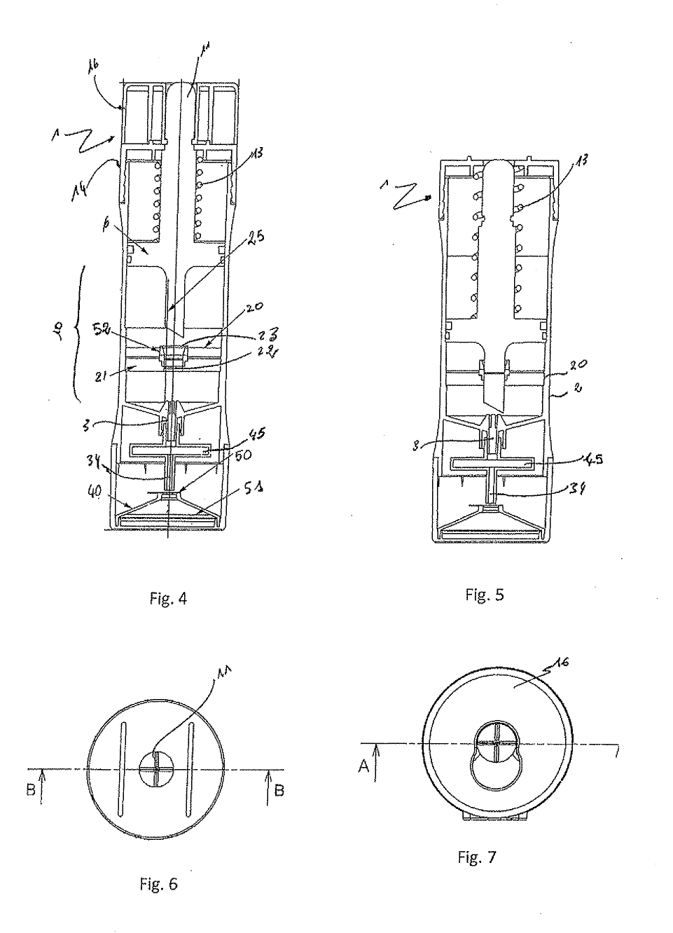

FIG. 4 is a diagrammatic axial section of an autonomous portable device for the automatic extemporaneous preparation of an active solution from two formulations, this device being in accordance with the invention and illustrated in a resting or storage position.

FIG. 5 is a section substantially equivalent to FIG. 1, showing the autonomous portable device according to the invention in the working position at the end of the sequence for preparing the active solution.

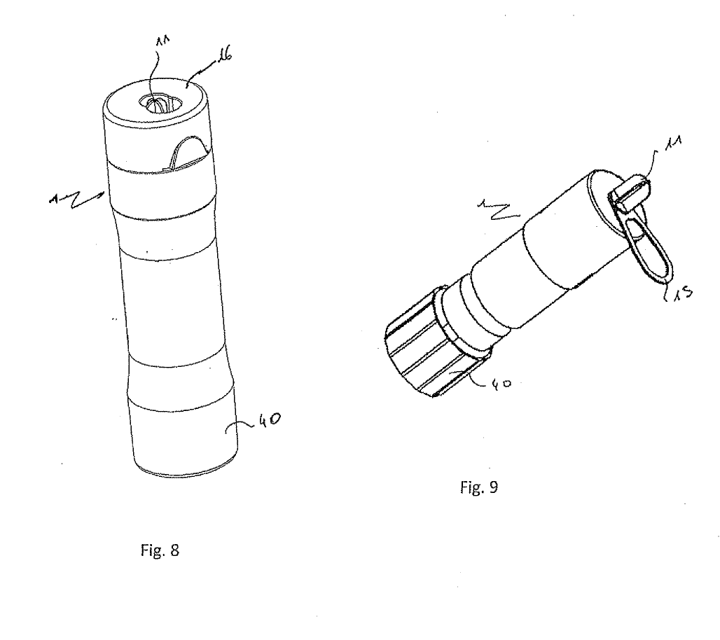

FIGS. 6 and 7 are views from above of the removable stopper masking the locking rod of the autonomous portable device as illustrated in FIGS. 4 and 5.

FIGS. 8 and 9 are views of assemblies of the autonomous portable device in a resting or storage position as illustrated in FIGS. 1 and 5.

An autonomous portable device for the automatic extemporaneous preparation of an active solution from formulations such as solution A and formulation B, denoted, as a whole, by reference 1 in FIGS. 1, 2, 4 and 5, comprises an elongated tubular body 2 which extends along an axis Δ. The tubular body 2 has a substantially cylindrical general shape, revolving about the axis Δ and has, at a distal end D, a head 3 in which a transfer channel 4 fits, said transfer channel having a passage section which is smaller than the passage section passage of the tubular body 2. On the opposite end from its distal end, the tubular body 2 has, at a proximal end P, a filling aperture 5. The device 1 also comprises a piston 6 which fits into the body 2 in such a way as to be able to slide translationally along the axis Δ. The piston 6 is, according to the example illustrated, fitted with two peripheral seals 7 and 8 which cooperate with the internal wall of the tubular body so as to ensure leaktight sealing between the latter and the piston while at the same time permitting the translation. The piston 6 then defines, in combination with the tubular body 2, a first compartment 10 for containment of the solution A. The piston 6 further bears a locking rod which extends on the opposite side to the distal end D through the filling aperture 5. The rod 11 has, remote from the piston 6, a peripheral locking groove 12, the function of which will subsequently become apparent.

The device 1 also comprises a spring 13 interposed between the piston 6 and a maintaining cap 14 which is fitted onto the tubular body 2 in such a way as to be translationally immobilized along the axis Δ. In order to maintain the piston in resting or storage position R as illustrated in FIG. 1, a removable locking tongue 15 is used, which is inserted into the groove 12 while being interposed between the rod and the cap 14. The tongue 15 prevents translation of the piston 6 under the effect of the spring 13 which is in a compressed state.

According to the embodiment represented in FIG. 4, the piston is maintained in the resting or storage position R as illustrated in FIG. 4, through the use of a removable locking cover 16 which is fixed in a removable manner on the cap 14 in a position which blocks and maintains the rod 11 and prevents translation of the piston 6 under the effect of the spring 13 which is in a compressed state.

The device further comprises a spacer 20 which is placed inside the first compartment 10, being located remotely from the distal end D and from the piston 6 when the latter is in the resting position R. As shown more particularly in FIG. 3, the spacer 20 is hole-punched and has, at its centre, a substantially cylindrical tube 21 with an axis Δ. The tube 21 is closed off, at upper and lower faces of the spacer, by cleavable protective seals 22 and 23 in such a way as to define a second containment compartment 21 for the formulation B. In order to perforate the protective seals 22 and 23, as will subsequently become apparent, the piston 6 comprises, at its face directed toward the distal end D, a perforating finger 25 which has an axis Δ and is aligned with the tube 21.

According to the example illustrated in FIGS. 4 and 5, the reservoir 21 can be closed off with a removable stopper 52.

The device 1 also comprises a hollow body 30 which delimits a retaining chamber 31 which communicates with the first compartment 10 by means of the transfer channel 4. According to the example illustrated, the hollow body 30 is fitted in a removable manner on the head 3 by means of a tubular collar 32. The hollow body 30 also comprises, on the opposite side from the transfer head 3, an outlet head 33 into which an outlet channel 34 fits.

According to the example illustrated in FIGS. 1 and 2, the device finally comprises a collecting tube 40, fitted in a removable manner onto the outlet head 33. The tube 40 is intended, as will subsequently become apparent, to collect the active solution expelled at the outlet channel 34.

According to the example illustrated in FIGS. 4 and 5, the collecting device 40 is in the shape of a funnel and has a cleavable protective seal 50 and a filling level 51.

The autonomous portable device 1 according to the invention, as thus formed, is used in the following way.

First of all, the formulation B is placed in the spacer, in the tube 21 which is closed off by the protective seals 22 and 23. In the context of the preparation of a solution of a hypothiocyanite, approximately 15 mg of a powder of lactoperoxidase and of a glucose oxidase will be stored in the tube 21 forming the second compartment.

Next, the spacer is fitted inside the tubular body 2 in such a way as to be substantially at the center of the first compartment 10. The transfer head 3 is, moreover, closed off by means of stopper that is not represented. The first compartment is then filled with a volume of formulation A that is sufficient for the spacer to be completely bathed in the solution when the axis Δ of the device is oriented vertically. According to the example illustrated, approximately 25 ml of the solution A will be stored in the first compartment, said solution A being a saline solution, comprising glucose, sodium thiocyanate and a buffer, pH 6.8 (50 mM KH₂PO_{4,} 1M NaOH).

According to the example illustrated, the first compartment 10 is only partially filled with solution A, such that there exists a volume of gas, such as air or a neutral gas, between the free surface of the solution and the piston 6.

Moreover, the maintaining cap 14, the piston 6 and the rod 11, and also the tongue 15 or the locking cover 16, are assembled with the spring 13 which is in a compressed state and maintained in this state by the insertion of the tongue 15 in the groove 12 or the attaching of the locking cover 16. This assembly, which forms manually triggered mixing and expelling means, is then fitted onto the tubular body 2 which has, for this purpose, two ergots 41 intended to engage with the locking grooves, which are not represented, of the maintaining cap 14 according to a bayonet locking system. Once this assembly has been prepared, the device is in a ready-to-use state in which it can be stored before use without any risk of detrimental alteration of the formulations A and B.

When the active solution must be obtained, the stopper of the transfer head 3 is removed and the body 30 is fitted onto the latter. The collecting tube 40 is further fitted onto the outlet head 33. The device 1 can then be placed on a support at the bottom of the tube 40, which will be of sufficient size to ensure the stability of the device 1.

The tongue 15 (or the locking cover 16) is subsequently removed, such that the rod 11 is released, allowing the spring 13 to apply a force on the piston 6 in such a way as to push it toward a working position T, as illustrated in FIG. 2. Under the effect of the pressure exerted by the spring 13, the piston 6 descends and the perforating finger 25 destroys the upper protective seal 23, thereby contributing to bringing the content of the second compartment 21 into contact with that of the first compartment 10, such that the reaction between the formulation A and the formulation B can begin. It should be noted that the passage section of the transfer channel 4 and the spring 13 are consequently proportioned in relation to the other elements of the device, in such a way that the flow through the transfer channel 4 occurs at a given speed. Thus, under the effect of the translation of the piston 6, the formulation A mixed with the formulation B undergoing reaction, flows into the retaining chamber 31 which, according to the example illustrated, further comprises a filter 45 interposed between the transfer channel 4 and the outlet channel 34. The filter 45 therefore acts according to a frontal filtration mode since it extends substantially perpendicular to the axis Δ of the transfer channel and to the direction of flow of the mixture of the formulations A and B. The retaining chamber 31 is therefore proportioned in such a way that the liquid resulting from the mixing of the solution A and of the formulation B flows through the outlet channel 34 only after a certain translation of the piston and therefore a minimum time of reaction between the formulation A and the formulation B. In the context of the preparation of a solution of hypothiocyanite ions, the stroke of the piston 6, the force of the spring 13, and the dimensions of the transfer channel 4 and of the retaining chamber 31 are selected in such a way that the active solution begins to flow through the channel 34 only after a period of time of more than 5 minutes has elapsed. It should be noted that the filter 45 also contributes to controlling the flow kinetics of the active solution. According to the example illustrated, the filter 45 is a frontal filter for ultrafiltration below 70 kD, and for example of the order of 50 kD. In order to enable filtration through such a filter, the spring 13 is preferably adapted such that the pressure of the mixture, measured at the internal face of the filter 45 inside the chamber 31, is greater than 5 bar or 5×10⁵ Pa.

At the end of the translation of the piston 6, the latter has reached its working position T, as illustrated in FIG. 2 in which it is pressing against the spacer 20. When the piston has reached its working position T, after a translation time of approximately 20 min, a volume of active solution of the order of 5 ml, which can then be used for daily treatment, has been collected in the tube 40.

Moreover, in order to increase the effectiveness of this active solution, it may also be envisioned to have, in the tube 40, a volume of a formulation in liquid or solid form C, which will mix with the active solution collected. In the present case, a lactoferrin solution or powdered lactoferrin is used.

Thus, after the device has been actuated, an active preparation ready to be administered, comprising 250 μM of OSCN⁻ ion, 2.4 mM of SCN⁻ ion and 2.6 mM of lactoferrin, is obtained. The preparation contains neither glucose oxidase, nor lactoperoxidase, nor hydrogen peroxide.

Of course, the device according to the invention can be used for the preparation of types of active solutions or of solutions that are useful in the analysis and/or diagnostics fields, other than that described above.

For example, the formulation A may be an isotonic solution and the solution B a freeze-dried product, the device making it possible both to be sure that the freeze-dried product is completely dissolved (control of dissolution time) through the type of filtration and the energy supplied by the spring, and to remove any solid particle from the solution that will be collected, enabling, for example, inhalation thereof or intravenous administration thereof.

1. An autonomous portable device for the storage of at least two formulations and for the automatic extemporaneous preparation of an active solution from the two formulations, said device comprising:
a first compartment for containment of a first formulation,
a second compartment for containment of a second formulation,
a retaining chamber which communicates at least with the first compartment by means of at least one transfer channel and which comprises at least one outlet channel for the active solution,
manually triggered mixing and expelling means suitable for, after triggering, automatically:
bringing the two formulations into contact,
and expelling, via the outlet channel, at least a part of the active solution at the end of a minimum reaction time.

2. The device as claimed in claim 1, wherein at least one of the formulations is in solid form.

3. The device as claimed in claim 1, wherein at least one of the formulations is in liquid form.

4. The device as claimed in claim 1, which comprises:
an elongated tubular body which comprises, at a distal end, a head into which the transfer channel is fitted and, on the opposite side from the head, at a proximal end, a filling aperture and which partly delimits at least the first compartment,
a hole-punched spacer which is placed inside the tubular body, remote from the distal end, and which delimits the second compartment closed off, at opposite faces of the spacer, by two cleavable protective seals,
a piston which can move translationally in the tubular body between a resting position and a working position and which bears, on the one hand, on a face directed toward the spacer and the distal end of the body, a perforating finger intended to perforate the protective seals of the second compartment, and, on the other hand, on a face directed toward the proximal end, a locking rod,
a spring which is interposed between the piston and a maintaining cap rigidly connected to the body in such a way as to push the piston toward its working position,
a locking means,
a hollow body which delimits the retaining chamber, which is connected to the transfer head and comprises, on the opposite side from the transfer head, an outlet head in which the outlet channel is fitted.

5. The device as claimed in claim 4, wherein the locking means is a removable tongue which is interposed between the locking rod and the cap in such a way as to maintain the piston in the resting position.

6. The device as claimed in claim 4, wherein the locking means is a removable cover which masks the locking rod and the cap in such a way as to maintain the piston in the resting position.

7. The device as claimed in claim 4, wherein the stiffness of the spring and the compression of the spring in the resting position of the piston, the stroke of the piston, the passage section of the transfer channel and the volume of the retaining chamber are adapted such that the expulsion of the active solution occurs starting from 5 min after withdrawal of the locking tongue.

8. The device as claimed in claim 1, which comprises a filter placed in the retaining chamber, being interposed between the transfer channel and the outlet channel in such a way as to provide a frontal filtration.

9. The device as claimed in claim 8, wherein the filter is suitable for providing an ultrafiltration below 200 kD.

10. The device as claimed in claim 8, wherein the filter is suitable for providing an ultrafiltration below 70 kD.

11. The device as claimed in claim 8, wherein the filter is suitable for providing an ultrafiltration at 50 kD.

12. The device as claimed in claim 8, wherein the filter is suitable for providing an ultrafiltration at 30 kD.

13. The device as claimed in claim 8, wherein the filter is suitable for providing an ultrafiltration at 10 kD.

14. The device as claimed in claim 8, wherein the spring is suitable for exerting a pressure, measured at the internal face of the filter, of between 1 and 7 bar.

15. The device as claimed in claim 1, wherein the first compartment is partially filled.

16. The device as claimed in claim 1, which comprises a removable tube for collecting the active solution, fitted at the outlet channel.

17. A collection or kit for the storage of two formulations and for the automatic extemporaneous preparation of an active solution, which comprises a device as claimed in claim 1 and wherein the first formulation in solution comprises at least one aqueous-phase oxidizable substrate capable of giving the hypothiocyanite and/or hypohalite ion.

18. The collection or kit as claimed in claim 17, wherein the first formulation in solution further comprises at least one reducible substrate.

19. The collection or kit as claimed in claim 17, wherein the solid or liquid second formulation comprises at least one enzymatic catalysis agent.

20. The collection or kit as claimed in claim 19, wherein the solid or liquid second formulation further comprises a supplementary enzyme system.

21. The collection or kit as claimed in claim 17, wherein the oxidizable substrate is constituted of at least thiocyanate or halide ions.

22. The collection or kit as claimed in claim 17, wherein the second formulation comprises at least one lactoperoxidase or one peroxidase.

23. The collection or kit as claimed in claim 18, wherein the reducible substrate is glucose.

24. The collection or kit as claimed in claim 20, wherein the supplementary enzyme system is a glucose oxidase.

25. The use of a device as claimed in claim 1, for the preparation of solutions which are useful in the analysis and/or diagnostics fields.

26. The use of a device as claimed in claim 1, for the preparation of purified and/or protein-free and/or microorganism-free solutions.
